(19) United States
(12) Patent Application Publication
(10) Pub. No.: US 2004/0223621 A1
(43) Pub. Date: Nov. 11, 2004

(54) Electronic auscultation kit

(76) Inventor: Birger Orten, Vettre (NO)

Correspondence Address: LADAS & PARRY LLP, 224 SOUTH MICHIGAN AVENUE, SUITE 1200, CHICAGO, IL, 60604, US

(21) Appl. No.: 10/257,503

(22) PCT Filed: Apr. 11, 2001
(86) PCT No.: PCT/NO01/00164

(51) Int. Cl. A61B 7/04; A61B 5/02; A61B 7/02

(52) U.S. Cl. 381/67; 181/131; 600/528

(57) ABSTRACT

A hand-held and hand-operated electronic stethoscope sensor module (1) constitutes the core of an equipment kit which in addition to the sensor module (1), comprises at least one extra equipment unit to be attached to the sensor module (1) optionally. The sensor module (1) is provided with interface means adapted to each one of the extra equipment units. Examples of cooperating pairs of extra equipment unit and interface means, are: wireless headset (9) and radio antenna (7) on the module (1); front piece (16) for machine part listening, and front listening surface (3) on the module (1); galvanic safety separation unit (23) and appurtenant connector on the module (1).

The present invention concerns electronic stethoscopy in general, and more particularly an equipment kit for electronic auscultation, in accordance with the preamble of the appended claim 1.

Electronic stethoscopes are previously known, see for example the previous Norwegian patents 300,250, “Auscultation apparatus” and 304,870, “Listening head for an electronic stethoscope” belonging to the present applicant. The first mentioned publication indicates the general structure of an electronic stethoscope, and the last mentioned publication relates to a listening head which in one embodiment may be constituted by a hand-held unit that may for instance transmit auscultation sounds by means of radio signals to a stationary unit nearby. In the first mentioned publication, the use of a headset is mentioned in connection with electronic stethoscopy, and there is also mention of a microphone unit to be put in direct contact with a body surface. Hence, the prior art contains a hand-held stethoscope sensor module having a connected headset, i. e. with a cable from the sensor module to the headset. The prior art also contains wireless transmission from a hand-held sensor module.

However, a hand-held electronic stethoscope sensor module of the type mentioned, can be used in other connections than the originally intended connection with a connected headset, and it is of interest to expand the possibilities of use in connection with more advanced forms of investigation, and in connection with recordal of investigation data and in connection with e.g. instruction or group work.

The present invention provides a solution to the problem in connection with such expansion of use possibilities, and does so by establishing an equipment kit of the type mentioned in the introduction, and which is characterized by the features appearing in the characterizing part of the appended claim 1. Preferred embodiments of the invention appear from the attached dependent claims.

In the following, the invention shall be illuminated in more detail by describing embodiment examples thereof, and it is at the same time referred to the appended drawings, in which

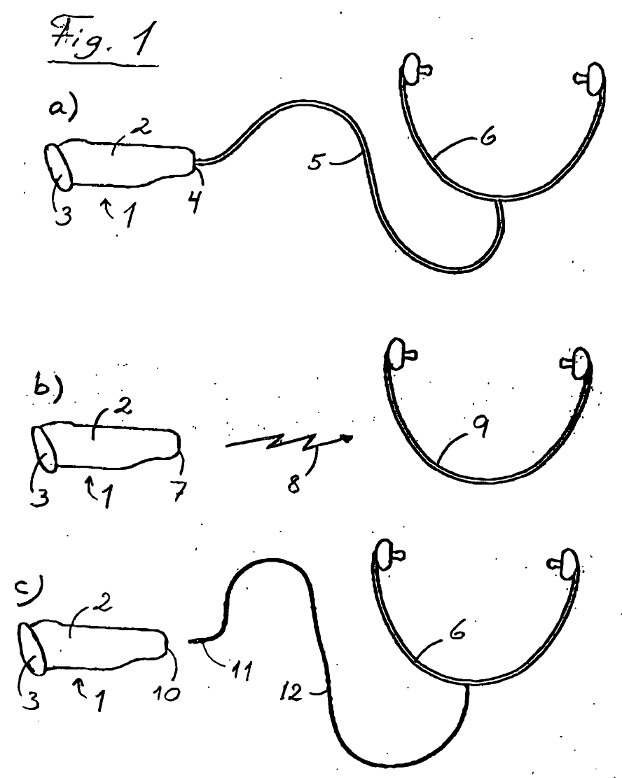

FIG. 1 shows possible basic parts of the equipment kit, as well as extra equipment providing a list ning possibility for an attending doctor or an operator,

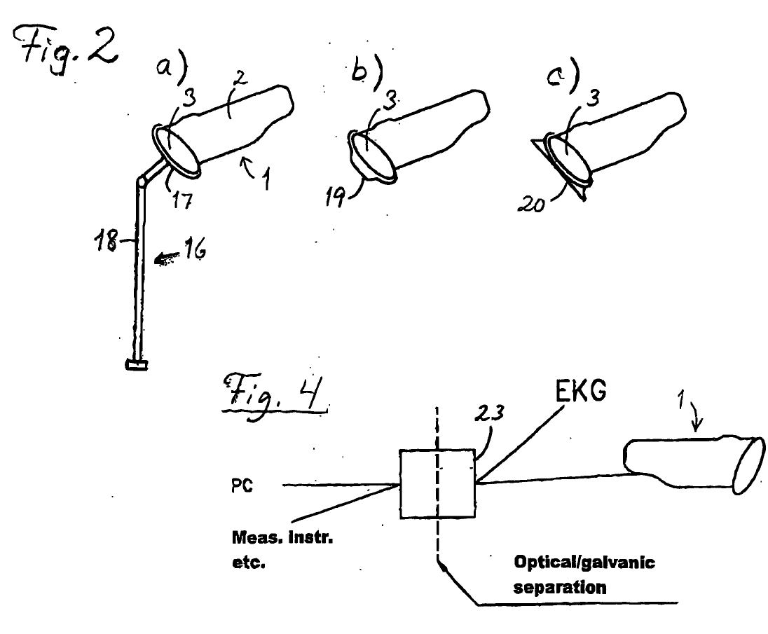

FIG. 2 shows certain types of extra equipment for use in special listening situations of medical type and other types,

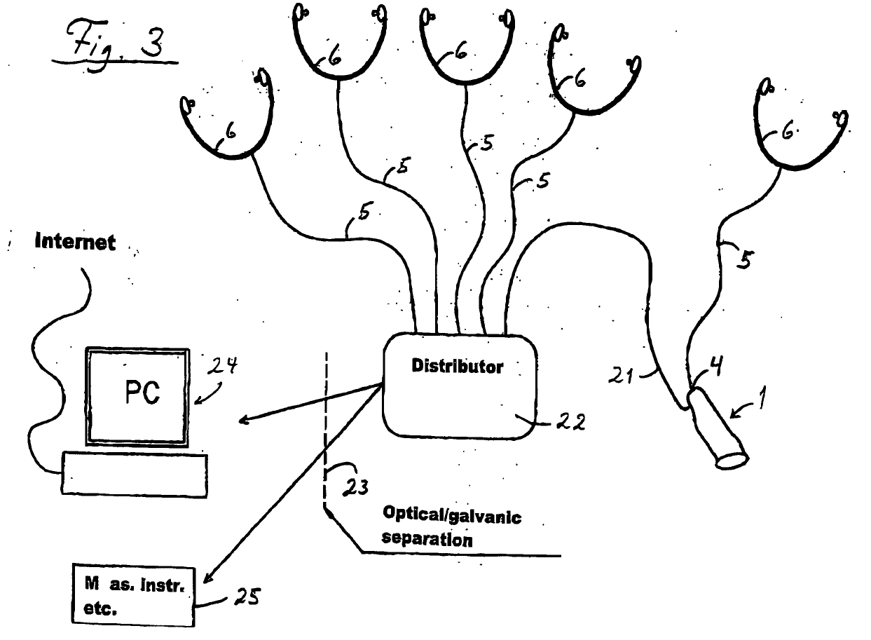

FIG. 3 shows extra equipment for co-listening and recording, and

FIG. 4 shows extra equipment for providing galvanic separation between the sensor module and mains operated recording equipment.

FIG. 1a shows a basic form in which a hand-held stethoscope sensor module 1 with a main body 2 and front listening surface 3 appear, with a headset 6 permanently connected via a signal line 5 and a permanent coupling 4. Hence, this complete apparatus shall be regarded as a basic unit, to which extras can be connected.

However, it is also possible to start from a less comprehensive basic form in an alternative version, i.e. a basic form such as appearing in FIGS. 1b and 1c, viz. a handheld module 1 without a permanent line coupling. In the version of FIG. 1b, the module 1 is equipped with a (preferably invisible) antenna 7, for transmission of a radio signal 8 to be received by a self-supporting headset 9 (i.e. comprising necessary power supply, receiver and amplifier for the headset loudspeakers). The antenna 7, constituting an interface toward the headset, may also in principle be replaced by a lamp for emitting modulated light, preferably invisible (infrared) light, or by an ultrasound transmitter, however short-wave signal transmission is easily stopped by obstacles, for example the body of the doctor/operator and other bodies. Therefore, radio signals are preferable.

With such a basic form, which appears also in FIG. 1c, approximately the same embodiment as constituted by the basic form in FIG. 1a, is obtainable by means of an extra consisting of a headset 6 with a signal line 12 that can be connected and disconnected to the module 1 by means of a connector 11 and corresponding connector 10, said corresponding connector 10 being defined as the module interface toward the headset.

So far, we have mentioned extra equipment of the type placed “behind the module” i.e. on the doctor/operator side. One of the extras 9 or 11+12+6, or for that matter both these extras, may be included in the equipment kit in accordance the invention.

Another type of extra that can be included in the equipment kit in accordance with the invention, is an extra to provide expanded possibilities for listening, i.e. changed listening situations. In FIG. 2a appears an extra 16 that is specially adapted for listening to machine or construction parts, including a rod or a tube 18, shown here in an articulated embodiment, and with an adaptation part (17) toward the listening surface 3. Such a rod must be made of a rigid and hard material for good transfer of sound/vibrations. An articulated, or non-articulated, rod 18 can be useful for listening analysis of machine parts that are difficult to access. It should be noted at the same time that stethoscopy/auscultation, in connection with this application, shall mean listening not only to living bodies, but also to machine parts, construction parts and other fields suitable for listening analysis. The listening surface 3 on the sensor module constitutes the module interface toward equipment 16.

In FIG. 2b appears an extra in the form of a front piece 19 with a downscaled listening area, and with a direct adaptation to the module listening surface 3. In certain connections, a surface to be subject to auscultation, is small, for instance on small children, and such an adaptation part may then be useful.

In FIG. 2c appears an opposite case, namely an extra 20 for picking up sound from a larger area than what can be covered by the listening surface 3 of the sensor module.

In FIG. 3 appears another extra equipment that can be included in the equipment kit, namely a distribution unit 22 for co-listening, to be used in education situations or in group work, and to which a series of headsets 6 can be connected by means of lines 5. In this embodiment, line 21 connects sensor module 1 and distribution unit 22. However, line 21 may just as well be replaced by a wireless connection between sensor module 1 and distribution unit 22, by means of built-in antennas in the two units. A connection by means of light signaling is also possible, as previously mentioned. The distribution unit 22 should contain an amplifier with regard to distributing signals to several line connected headsets. (Of course it is also possible to envisage wireless transmission to every headset from the distribution unit 22, or possibly directly from the sensor module 1 to a plurality of self-supporting headsets.)

In the same drawing appears also a possibility for further connection from the distribution unit 22 to a PC 24 or measurement instruments 25. If line conn ctions are used, it is important, from safety reasons, that there is a galvanic separation unit on the signal line between distribution unit 22, that may be battery operated, and mains operated equipment 24, 25. The galvanic separation 23 may be implemented by means of for instance optoisolator technology.

FIG. 4 shows the case in which a handheld sensor module 1 is line connected directly to e.g. a PC or to measurement instrumentation. If these instruments are mains operated, it is, in the same manner as in the above mentioned case, important from safety reasons, that there is a galvanic safety separation unit 23 on the signal line connection between the sensor module and the PC/measurement instrument. It is also possible to safeguard EKG sensing equipment with the same separation unit. Such a galvanic separation unit 23 may also be included in a topical equipment kit.

It is also possible to have direct wireless connection from the sensor module 1 to a separate signal processing unit. i.e. by means of radio signals, light or ultra sound. Such a signal processing unit for wireless reception of a signal from the sensor module, may also be included in the equipment kit.

An equipment kit in accordance with the invention will naturally be supplied e.g. in a practical suitcase, and a kit can be adapted specially for a certain purpose. For instance, one may plan a kit custom-made for listening to machine parts and to be used when teaching such listening to others, then containing a sensor module 1 as a basic part, and additionally a distribution unit 22, for example five headset pieces 6 with line 5 and connector, and connecting line 21 between module and distribution unit, and for example three front pieces 16 with differently designed pipes/joints/lengths for accessing and listening to machine parts that are difficult to access.

A simple equipment kit for a general practitioner may comprise a wireless sensor module 1 as a basic part, and as extra equipment, a wireless headset 6 and a wireless signal processing unit 22, as well as surface area adaptation parts 19, 20. Possibly, the kit may comprise a sensor module with fixedly attached headset (1+5+6) as the basic part, and as extras, only surface adaptation units 19, 20.

For specialists/hospitals one may include special front attachments, adapted to certain areas of a patient body, possibly also for inner cavities and orifices of the body, and distribution units 22 with several headsets 6 will also be included as parts of natural equipment kits. A separation unit 23 should also be included. In a hospital situation, a relatively long listening situation with automatic recording of progress may become important, and in such a connection, a kit should also include an attachment strap for placing the stethoscope sensor module steadily on the patient body. The sensor module may then be equipped with special attachment details adapted to the attachment strap.

1. An electronic auscultation equipment kit, including a hand-held and hand-operated electronic stethoscope sensor module, possibly having a fixedly mounted headset with a line, characterized in that
said kit further comprises at least one extra equipment unit to be attached to said sensor module optionally, and that
said stethoscope sensor module is provided with interface means adapted to every said extra equipment unit.

2. The equipment kit of claim 1, characterized in that said at least one extra equipment unit is selected among a group which comprises
front pieces for listening area down-scaling, to be used for example on small children,
front pieces having a rigid extension rod or pipe, to be used in listening for instance to machine parts,
headsets of a wireless type,
headsets with line and connector,
wireless signal processing unit,
distribution unit for headset co-listening,
galvanic safety separation unit between said sensor module and another electrical apparatus that is mains connected, for instance a PC or measuring equipment.

3. The equipment kit of claim 1, characterized in that the sensor module interface means are selected among a group comprising
connector for a line from a headset,
antenna for radio connection to wireless headsets,
lamp for light connection to wireless headsets,
antenna for radio connection to signal processing unit,
lamp for light connection to a signal processing unit,
connector for a line to a distribution unit for headset co-listening,
antenna for radio connection to a distribution unit for headset co-listening,
lamp for light connection to a distribution unit for headset co-listening,
connector for a line to a galvanic safety separation unit.

4. The equipment kit of claim 1, characterized in that a front listening surface on the sensor module constitutes one of the module interface means.
